(19) United States
(12) Patent Application Publication
(10) Pub. No.: US 2024/0424459 A1
(43) Pub. Date: Dec. 26, 2024

(54) MASSLESS MIXING SYSTEM AND METHOD OF USE

(71) Applicant: Nob Hill Therapeutics, Inc., Albuquerque, NM (US)

(72) Inventor: James R. Meckstroth, Cary, NC (US)

(73) Assignee: Nob Hill Therapeutics, Inc., Albuquerque, NM (US)

(21) Appl. No.: 18/708,244

(22) PCT Filed: Nov. 8, 2022
(86) PCT No.: PCT/US2022/049294

§ 371 Date: May 8, 2024

Related U.S. Application Data
(60) Provisional application No. 63/277,162, filed on Nov. 8, 2021.

(51) Int. Cl. B01F 33/452; B01F 35/00; B01F 35/32

(52) U.S. Cl. CPC B01F 33/452; B01F 35/187; B01F 35/3204

(57) ABSTRACT

A massless mixing system comprising: a power source, a massless drive, wherein the massless drive comprises a collar forming a channel, conductive wire; and a first housing in communication with the channel. The massless mixing system can further comprise a magnetic responsive element, a circuit, a second housing, or a combination thereof. The massless drive can comprise a solenoid drive, a brushless direct current drive, other massless drive configurations, or a combination thereof. A method of massless mixing comprising providing a material to be mixed; disposing a portion of the material into a mixing vessel; contacting the material with a magnetic responsive element; programming a software to apply power to a massless drive; applying power to the massless drive; and applying an electromagnetic field to the magnetic responsive element.

### CROSS-REFERENCE TO RELATED APPLICATIONS

This application claims priority to and the benefit of the filing of U.S. Provisional Patent Application No. 63/277,162, entitled “MASSLESS MIXING SYSTEM AND METHOD OF USE”, filed on Nov. 8, 2021, and the specification and claims thereof are incorporated herein by reference.

### BACKGROUND OF THE INVENTION

### Field of the Invention (Technical Field)

Embodiments of the present invention relate to a massless mixing system, including a dry powder nebulizer comprising a massless mixing system.

### Description of Related Art

Laboratory and industrial mixing have been limited by uncontrolled methods. Mixing typically occurs in solutions or slurries where mixing is performed around a single axis. Examples of single-axis mixing include a cement mixing truck, or a benchtop stir plate. Other forms of mixing include vibrational agitation or shaking, such as with a sonicator or shaker, respectively. Regardless of the method, mixing requires a user to apply a rotation per minute, frequency, or other parameter to a material, which can include, for example, a solution or slurry, for a known period of time. The material itself is manipulated by an external force, which can include a movable surface, vibrating solvent, or a magnetic responsive element (“MRE”), e.g., a stir bar.

Current mixing technologies are subject to a number of limitations. The material may not be evenly mixed, for example when a rotating cement truck cylinder unevenly applies force to wet cement. The material may be viscous or heterogenous and therefore require extended mixing times. Extended mixing times may be required, for example, to disrupt clumped powder in a solvent. An MRE is often fixed in constant direct contact to the bottom of a mixing vessel by magnetic force and rotated by a magnetic field below the MRE. The MRE applies force to the material to mix it. However, the magnetic field may be too weak to move the MRE in heterogenous mixtures requiring a solid to be mixed into a solution because the solid may cover the MRE and stop its rotation. This limits the amount of solid that may be added to a solution, particularly under benchtop laboratory conditions where the strength of the magnetic field is restricted by the power and size of stir plates. The inefficiencies and drawbacks of current mixing technology result in increased power and time required to mix materials.

Nebulizers have been more recently developed to deliver medication as a fine mist. Nebulizers employ electric power from a battery or external power source to convert a liquid or powder into an aerosol that then flows into a mouthpiece or mask worn by the user. Aerosol delivery allows a user to ingest the medication without the mechanical work of deep or rapid inhalation. However, nebulizers are often bulky, noisy, and require a longer period of time for the medication dose to be administered relative to an inhaler.

Embodiments of the present invention address the issues found with industrial or laboratory mixing and the limitations of inhalers and nebulizers. Embodiments of the present invention allow precise mixing of solids, liquids, and gases using controlled MRE movement. Embodiments also allow a precise dosage of medication to be rapidly and fully dispersed and converted to an aerosol and to be delivered to a user with limited mechanical assistance from the user while achieving desired lung deposition.

### BRIEF SUMMARY OF EMBODIMENTS OF THE PRESENT INVENTION

Embodiments of the present invention relate to a massless mixing system having a power source; a mixing vessel; a magnetic responsive element at least partially disposed within the mixing vessel; and a massless drive in communication with the mixing vessel, wherein the massless drive comprises: a collar forming a channel; and a conductive wire at least partially disposed around the collar. In another embodiment, the massless drive applies an electromagnetic field onto the mixing vessel. In another embodiment, the massless drive is at least partially disposed around the mixing vessel. In another embodiment, the massless drive is at least partially disposed beneath the mixing vessel. In another embodiment, the massless drive comprises a solenoid. In another embodiment, the massless drive comprises a multi-current multi-phase drive. In another embodiment, the massless drive comprises a plurality of massless drives controlling a vertical and a horizontal movement of the magnetic responsive element.

In another embodiment, the massless mixing system further comprises a circuit. In another embodiment, the circuit controls a power supplied to the massless drive. In another embodiment, the massless mixing system further comprises a first housing at least partially disposed around the massless drive. In another embodiment, the massless mixing system further comprises a second housing at least partially disposed above the mixing vessel. In another embodiment, the massless mixing system further comprises a filter. In another embodiment, the filter is disposed above the mixing vessel. In another embodiment, the massless mixing system further comprises a programmable software to control a flow of electricity through said massless mixing system.

Embodiments of the present invention also relate to a method of massless mixing, the method comprising: providing a material to be mixed; at least partially disposing a portion of the material into a mixing vessel; contacting the material with a magnetic responsive element; programming a software to apply power to a massless drive; applying power to the massless drive; and applying an electromagnetic field to the magnetic responsive element. In another embodiment, the method further comprises contacting the material with a filter. In another embodiment, the method further comprises rotating the magnetic responsive element. In another embodiment, the method further comprises moving the magnetic responsive element in a vertical direction. In another embodiment, the method further comprises moving the magnetic responsive element in a horizontal direction. In another embodiment, the electromagnetic field comprises a plurality of electromagnetic fields each applying a different force.

Objects, advantages and novel features, and further scope of applicability of the present invention will be set forth in part in the detailed description to follow, taken in conjunction with the accompanying drawings, and in part will become apparent to those skilled in the art upon examination of the following, or can be learned by practice of the invention.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more embodiments of the invention and are not to be construed as limiting the invention. In the drawings:

FIG. 1 is an illustration showing the electromechanical differences between a standard rotating permanent magnet and an embodiment of the present invention;

FIGS. 2A, 2B, 2C, and 2D are drawings which illustrate embodiments of a massless mixing systems at different sizes;

FIG. 3 is a photo showing a top-down view of an embodiment of a massless mixing system disposed around a cylindrical powder chamber and a plurality of MREs;

FIG. 4 is a flow chart conceptually showing the structure and function of an embodiment of a massless mixing system, including deagglomeration of a dry powder dose container;

FIGS. 5A and 5B are illustrations showing a dry powder nebulizer, positioned in the hand of a user, and which is formed using a massless mixing system according to an embodiment of the present invention;

FIG. 6 is a drawing showing a rear view of a dry powder nebulizer comprising an embodiment of a massless mixing system;

FIG. 7 is a drawing showing a front view of a dry powder nebulizer comprising an embodiment of a massless mixing system;

FIG. 8 is a drawing showing a cutaway of a dry powder nebulizer comprising an embodiment of a massless mixing system comprising a solenoid drive;

FIG. 9 is a drawing showing a cutaway of a dry powder nebulizer and MRE housing comprising an embodiment of a massless mixing system having a solenoid drive;

FIG. 10 is a drawing showing a cutaway of a dry powder nebulizer comprising an embodiment of a massless mixing system having a multi-coil electro-magnet with an excitation drive;

FIG. 11 is a drawing showing a cutaway of a dry powder nebulizer and MRE housing comprising an embodiment of a multi-coil electro-magnet with an excitation drive;

FIG. 12 is a drawing showing air flow through a cutaway of a dry powder nebulizer and mixing compartment comprising an embodiment of a massless mixing system having a solenoid drive;

FIG. 13 is a drawing showing an embodiment of a solenoid drive;

FIG. 14 is a drawing showing an embodiment of a multi-coil electro-magnet with an excitation drive;

FIG. 15 is a drawing showing an embodiment of magnetic poles orientation and the excitation phases to drive MRE motion for powder agitation including dispersing of a multi-coil electro-magnet drive;

FIG. 16 is a graph for copper wire showing magnetic strength at the wire coil surface vs. wire gauge for solenoid and multi-coil electro-magnet drive (labeled BLDC style) with a 3.6 volt excitation voltage;

FIG. 17 is a graph for copper wire showing current vs. wire gauge for solenoid and multi-coil electro-magnet drive (labeled BLDC style) with a 3.6 volt excitation voltage;

FIG. 18 is a drawing showing electronic components of a dry powder nebulizer comprising an embodiment of a massless drive system;

FIG. 19 is a drawing showing an exploded view of electronic components of a dry powder nebulizer having a massless drive system according to an embodiment of the present invention;

FIG. 20 is a drawing showing an embodiment of a massless mixing system comprising an axial-flux massless drive disposed beneath a mixing vessel; and

FIGS. 21A, 21B, and 21C are drawings showing an embodiment of driver setup for a massless mixing system wherein the driver setup comprises a 3-phase connection.

### DETAILED DESCRIPTION OF THE INVENTION

Embodiments of the present invention are directed to a massless mixing system comprising: a power source, a massless drive, wherein the massless drive comprises a collar forming a channel, conductive wire; and a first housing in communication with the channel. The massless mixing system can further comprise one or more magnetic responsive elements, a circuit, a second housing, or a combination thereof. The MRE can be at least partially disposed within the first housing. The massless drive can be at least partially disposed within the second housing. The massless drive can comprise a solenoid drive, a multi-coil multi-phase drive, other massless drive configuration, or a combination thereof.

Embodiments of the present invention are also directed to a method of mixing using a massless mixing system comprising: at least partially disposing a material into a housing, wherein the housing is in communication with a channel of a massless mixing system; contacting the material with one or more MREs; setting the mixing parameters for the massless mixing system; and applying a varying excitation current to the massless mixing system. The massless drive can comprise a solenoid drive, a multi-coil multi-phase drive, other massless drive configuration, or a combination thereof.

Embodiments of the present invention are also directed to a dry powder nebulizer comprising: a power source; a circuit; a massless drive, wherein the massless drive comprises a collar forming a channel, a conductive wire; a first housing in communication with the channel; a second housing; a filter in communication with the first housing and an outlet; and an air inlet. The dry powder nebulizer can further comprise one or more MRE. The massless drive can be at least partially disposed within the second housing. The massless drive can comprise a solenoid drive, a multi-coil multi-phase drive, or a combination thereof.

Embodiments of the present invention are also directed to a method of nebulizing dry powder comprising: at least partially disposing a material into a first housing, wherein the first housing is in communication with a channel of a massless mixing system; contacting the material with one or more MRE; flowing air into the second housing; setting the mixing parameters for the massless mixing system; applying a varying excitation current to the massless mixing system. The method can further comprise contacting the MRE to a filter. The method can further comprise contacting the MRE and material with a mesh to form a nebulized powder and flowing the nebulized powder into a second housing.

Embodiments of the present invention can provide enhanced emitted dose performance and medicine delivery to the lungs compared to other active pharmaceutical ingredient (“API”) treatments using a nebulizer or inhaler. Embodiments can also provide faster, more precise, and/or more thorough mixing, including powder dispersing compared to other mixing devices or methods. Optionally, embodiments can be used to mix materials including, but not limited to, solids, liquids, solutions, gases, homogenous mixtures, heterogenous mixtures, slurries, muds, powders, gels, pastes, non-Newtonian fluids, Newtonian fluids, or a combination thereof. The massless mixing system can be applied to non-pharmaceutical production fields, including, but not limited to, laboratory experimentation, for example for benchtop stir plates, industrial mixing, food or agricultural preparation, or construction.

The massless mixing system can generate magnetic drive profiles attuned to specific API's or powder dosage formulations and therapeutic treatments of interest. The massless mixing system can be programmed using a circuit, programmable software, artificial intelligence, or a combination thereof. The massless mixing system can mix a material by moving one or more MRE. The MRE can be moved by an electromagnetic field. The MRE can be moved horizontally, vertically, rotationally, diagonally, or in any other direction in a two or three-dimensional space. The MRE can be moved by pushing, pulling, and/or rotating the MRE using an electromagnetic field.

Embodiments of the present invention are directed to a massless mixing system having: a power source, a massless drive, wherein the massless drive has a collar forming a channel, a conductive wire; and a first housing in communication with the channel. The massless mixing system can further comprise an MRE, a circuit, a second housing, or a combination thereof. The MRE can be at least partially disposed within the first housing. The massless drive can be at least partially disposed within the second housing. The massless drive can comprise a solenoid drive, a multi-coil multi-phase drive, other massless drive configuration, or a combination thereof. The solenoid drive can comprise a collar forming a channel and conductive wires at least partially disposed around the collar. The multi-coil multi-phase drive can comprise a collar forming a channel and one or more bobbins attached to the collar. The conductive wire can be at least partially disposed around the one or more bobbins. The first housing is preferably in communication with the channel when it is at least partially disposed above the channel, at least partially disposed below the channel, or at least partially disposed within the channel. The first housing is also preferably in communication with the channel when an electromagnetic field generated by the massless drive penetrates the first housing.

Embodiments of the present invention are also directed to a method of mixing using a massless mixing system comprising: at least partially disposing a material into a housing, wherein the housing is in communication with a channel of a massless mixing system; contacting the material with one or more MRE; setting the mixing parameters for the massless mixing system; applying varying current to the massless mixing system; and applying a magnetic field to the MRE. The massless drive can comprise a solenoid drive, a multi-coil multi-phase drive, other massless drive configuration, or a combination thereof. The method can further comprise evaluating the mixing and adjusting mixing parameters during the mixing of the material. The method can further comprise transmitting mixing data to an external location. The location can be a “smart” phone, computer, server, or data storage unit. Mixing parameters can include, but are not limited to, the current and/or voltage applied to the massless drive, the sequence and timing of applying current and/or voltage to one or more massless drives, and the direction and strength of the magnetic field applied to the MRE.

Embodiments of the present invention are also directed to a dry powder nebulizer comprising: a power source; a circuit; a massless drive, wherein the massless drive comprises a collar forming a channel, a conductive wire; a first housing in communication with the channel; a second housing; a filter in communication with the first housing and an outlet; and an air inlet. The dry powder nebulizer can further comprise one or more MRE. The first housing can comprise a plurality of air inlets. The massless drive can be at least partially disposed within the second housing. The massless drive can comprise a solenoid drive, a multi-coil multi-phase drive, or a combination thereof. The solenoid drive can comprise a collar forming a channel and conductive wires at least partially disposed around the collar. The multi-coil multi-phase drive can comprise a collar forming a channel and one or more bobbins attached to the collar. The conductive wire can be at least partially disposed around the one or more bobbins. The first housing is preferably in communication with the channel when it is at least partially disposed above the channel, at least partially disposed below the channel, or at least partially disposed within the channel. The first housing is also preferably in communication with the channel when an electromagnetic field generated by the massless drive can penetrate the first housing. The dry powder nebulizer can further comprise a mouthpiece.

Embodiments of the present invention are also directed to a method of nebulizing dry powder comprising: at least partially disposing a material into a first housing, wherein the first housing is in communication with a channel of a massless mixing system; contacting the material with one or more MRE; flowing air into the second housing; setting the mixing parameters for the massless mixing system; applying varying current to the massless mixing system; applying a magnetic field to the MRE; and contacting the MRE and material with a mesh. The method can further comprise contacting the MRE and material with a mesh to form a nebulized powder and flowing the nebulized powder into a second housing. The method can further comprise evaluating the mixing and adjusting mixing parameters during the mixing of the material. The method can further comprise transmitting mixing data to an external location. The location can be a “smart” phone, computer, server, or data storage unit. Mixing parameters can include, but are not limited to, the current and/or voltage applied to the massless drive, the sequence and timing of applying current and/or voltage to one or more massless drives, and the direction and strength of the magnetic field applied to the MRE.

As used throughout the application, the terms “mixing” and/or “mix” means perturbing, agitating, deagglomeration, dispersing, disturbing, moving, manipulating, vibrating, stirring, or any other manners of applying a force to a material to change the arrangement of its particles, molecules, and/or components.

As used throughout the application, the term “magnetic responsive element (‘MRE’)” means any object or material that can be moved by a magnetic field. The MRE can comprise one or more of a stir bar, a polarized metal, a dipole, or combination thereof.

As used throughout the application, the term “filter” means any material that prevents particles greater than a given size from passing through it. The material can comprise a mesh, a porous membrane, a grate, a perforated sheet, a selective membrane, a porous network, a size-selective column, or any other material that prevent the passage of material based on size.

Although the application refers to a “massless” mixing system, it is to be understood that portions of the system do comprise a mass, including but not limited to the MRE, reference to the system as being “massless” is in reference to the massless connecting structure between the electromagnetic drive system and the MRE or material being mixed.

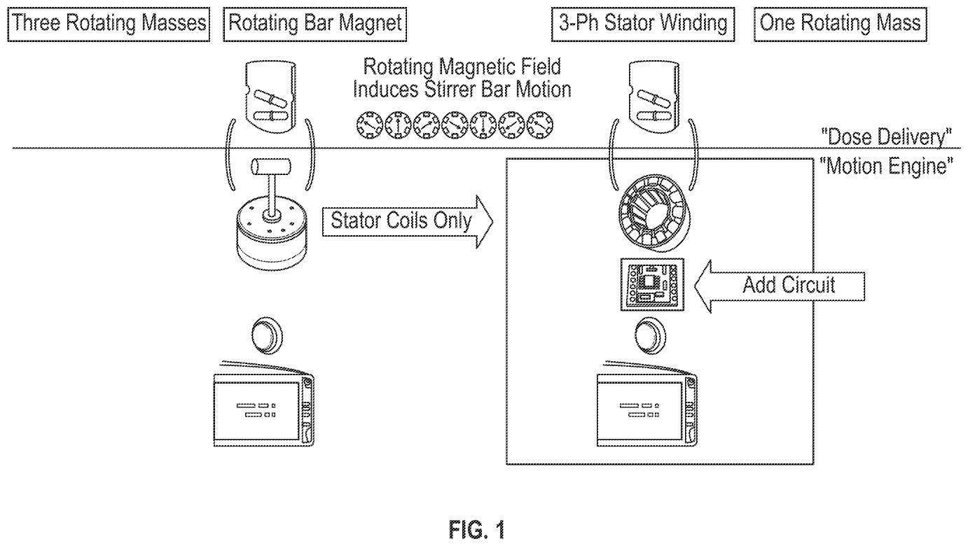

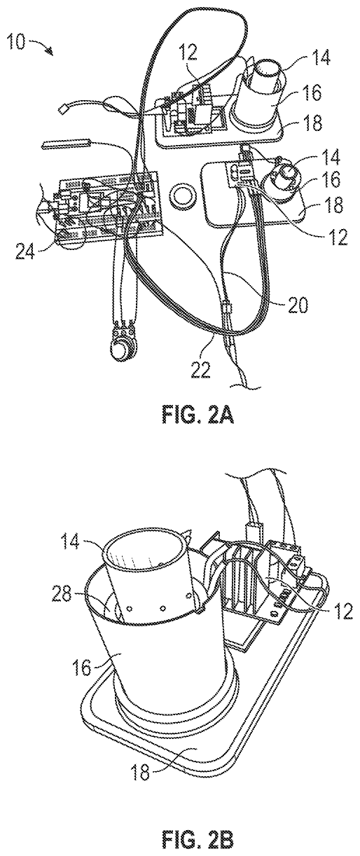

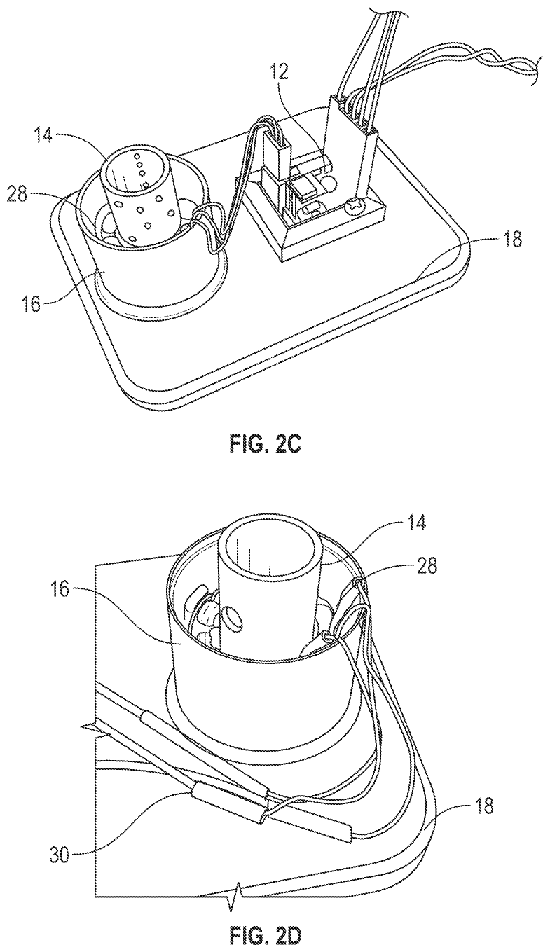

Turning now to the figures, FIG. 1 shows a comparison of a standard rotating permanent (bar) magnet and an embodiment of the massless drive of the present invention. FIGS. 2A to 2D illustrate embodiments of connected massless mixing systems 10 controlled by circuit 24. Each mass mixing system is run by controllers 12 and comprises mixing vessel 14, outer housing 16, base plate 18, outer collar 28, and one or more conductors 30. Conduit 22 provides power to connected massless mixing systems 10 and one or more conduits 22 facilitate communication between a massless mixing system and circuit 24.

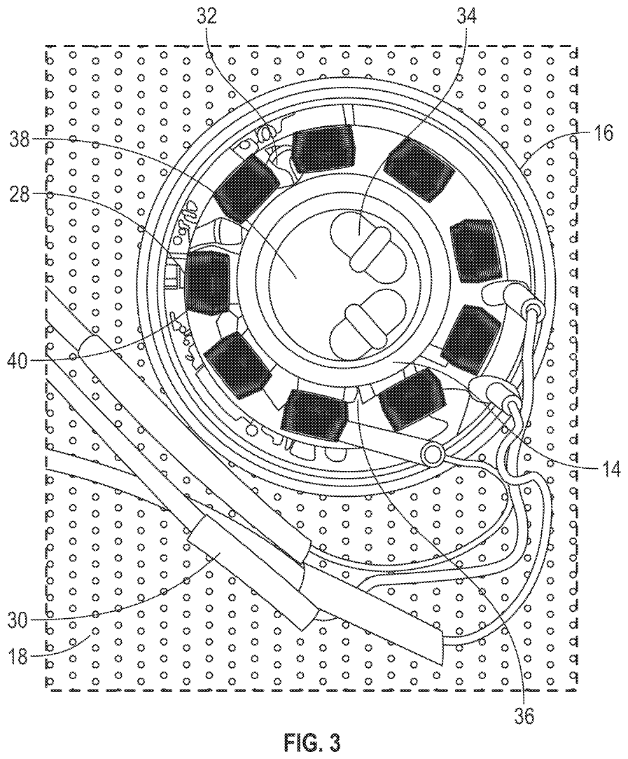

FIG. 3 shows a detailed view of an embodiment of massless mixing systems. MREs 34 are disposed within mixing vessel 14. Mixing vessel 14 is preferably at least partially disposed within multi-coil multi-phase drive 32. Multi-coil multi-phase drive 32 is preferably at least partially disposed within outer housing 16 and is powered by one or more conductors 30. Multi-coil multi-phase drive 32 preferably comprises outer collar 28, a plurality of bobbins 40, and inner collar 36.

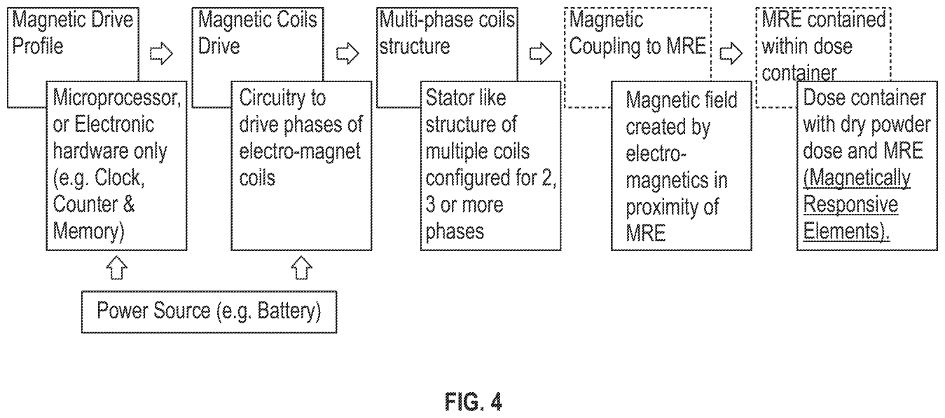

FIG. 4 describes the structure and function of an embodiment of a massless mixing system of a dry powder dose container. Electro-magnet coils are controlled by a microprocessor to control the movement of an MRE within a container.

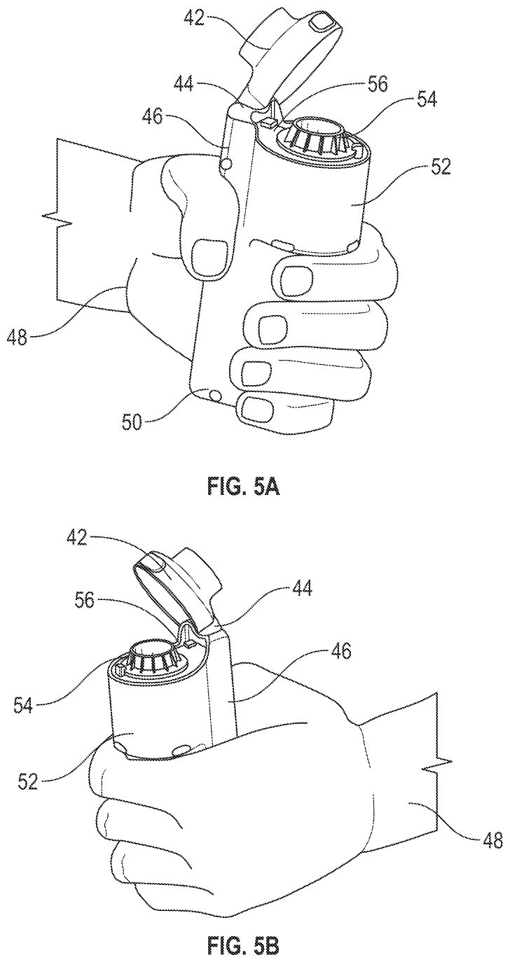

FIGS. 5A and 5B illustrate the position of a dry powder nebulizer comprising an embodiment of a massless mixing system in user's hand 48. A mouthpiece connects to opening of hinged top piece 42 which is preferably attached to rear housing 46 by swivel joint 44. Other connections and configurations could be used and would provide desirable results, including for example, a mouthpiece connecting to mouthpiece adapter 54 and hinged top piece 42 being removably positionable. Rear housing 46 is preferably connected to power housing 50, which can include for example a battery compartment, and massless drive system housing 52. Massless drive system housing 52 is preferably attached to cover plate 56. Mouthpiece adapter 54 is at least partially disposed within cover plate 56 and massless drive system housing 52.

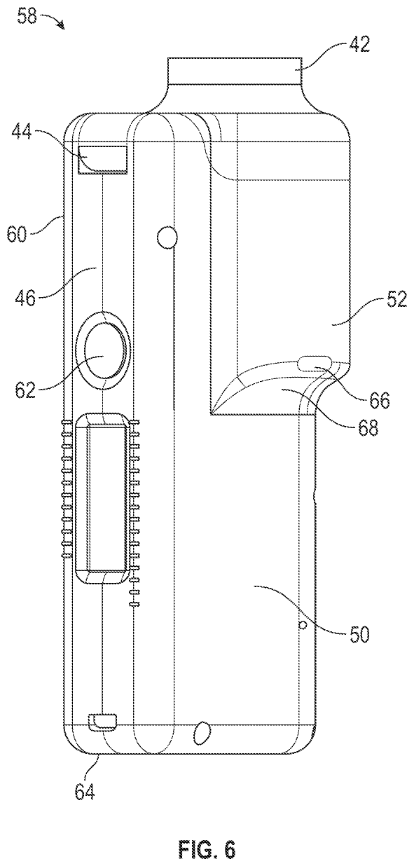

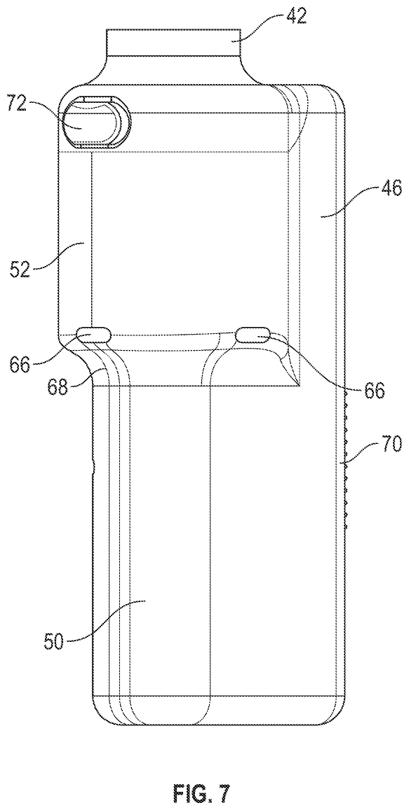

FIGS. 6 and 7 illustrate a rear and front view, respectively, of dry powder nebulizer 58 comprising an embodiment of a massless mixing system. Activation button 62 is partially disposed within rear housing 46. Although the size and dimensions of dry powder nebulizer 58 can be varied to meet any desired application, in one embodiment, rear housing 46 preferably has height 60 of about 145 millimeters (“mm”) and width 64 of about 30 mm. Power housing 50 preferably has width 64 of approximately 55 mm. Massless drive system housing 52 comprises air inlet channels 66 to allow air to flow into dry powder nebulizer 58. Massless drive system housing 52 also comprises grip surface 68 to assist a user in handling dry powder nebulizer 58. Hinged top piece 42 preferably comprises button 72 to release hinged top piece 42.

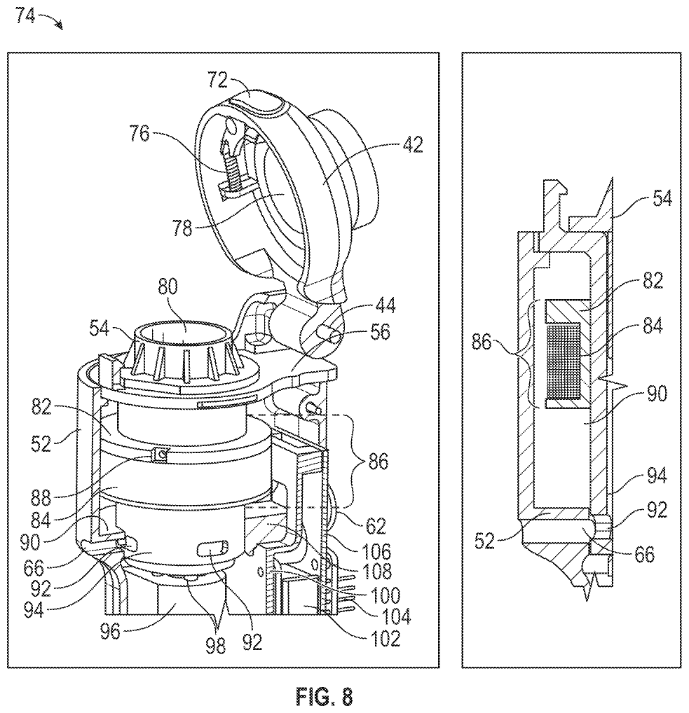

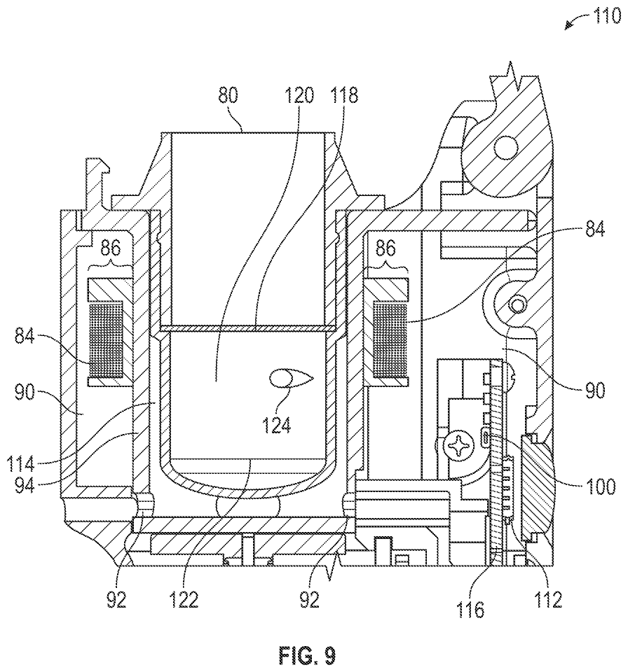

FIG. 8 illustrates a cutaway of a dry powder nebulizer 74 comprising an embodiment of a massless mixing system. Hinged top piece 42 comprises button 72 controlled by release mechanism 76. Nebulized material exits hinged top piece 42 through cavity 78. Mouthpiece adapter 54 forms cavity 80 to allow nebulized medication to flow out of dry powder nebulizer 74. Solenoid drive 86 is preferably disposed around interior housing 94. Solenoid drive 86 comprises collar 82, and conductive wiring 84 and is locked into position-for example via a fastener, which can include but is not limited to screw 88. The position of solenoid drive 86 can be adjusted along interior housing 94. Although the length can be varied to meet any desired application in one embodiment, solenoid drive 86 can be adjusted along a distance of about 17 mm. Interior housing 94 is preferably disposed in cavity 90 of massless drive system housing 52. Interior housing 94 comprises air inlets 92. Optionally, interior housing 94 can be in communication with permanent magnet 98. Interior housing 94 is preferably also in communication with conduit 108. Inner chassis plate 100 is attached to outer chassis plate 106. Battery 102 is disposed between inner chassis plate 100 and outer chassis plate 106. Activation button 62 is attached to a rear housing and display 104 is attached to outer chassis plate 106. Turning to FIG. 9 showing a cutaway of dry powder nebulizer 110, activation switch 112 is attached to outer chassis plate 106 (see FIG. 8). Mixing compartment 120 is disposed within cavity 114 of interior housing 94 and is in communication with filter 118 and cavity 80. Filter 118 is disposed between mixing compartment 120 and mouthpiece adapter 54 (see FIG. 8). Mixing compartment 120 comprises cavity 122 to mix material and orifice 124 to allow air to enter mixing compartment 120.

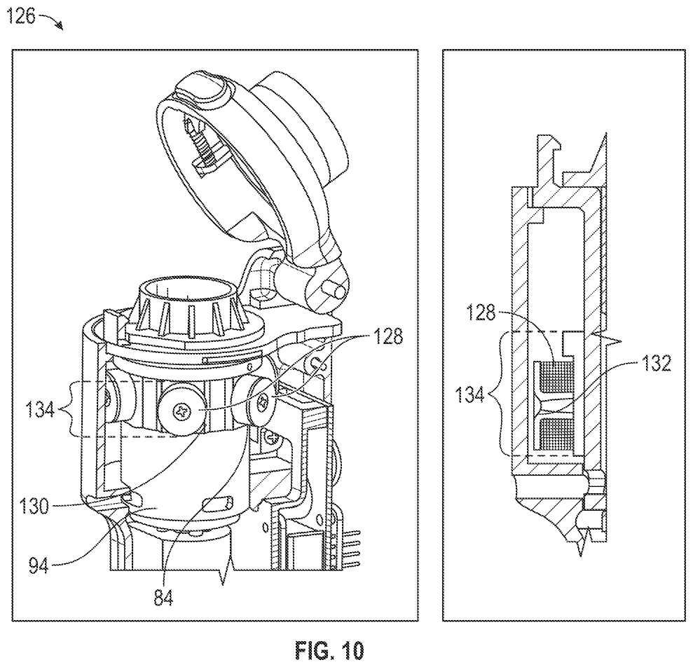

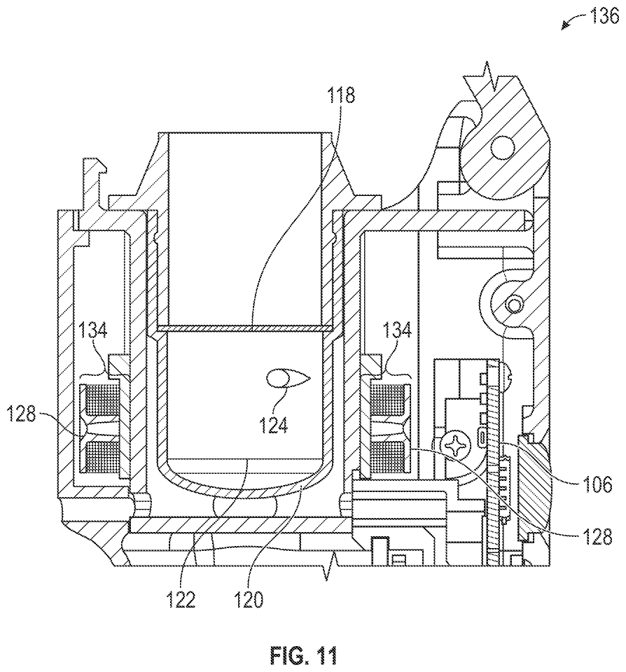

FIGS. 10 and 11 illustrate cutaways of a dry powder nebulizer 126 comprising a massless mixing system according to an embodiment of the present invention. Multi-coil multi-phase drive 134 is disposed around interior housing 94. Multi-coil multi-phase drive 134 comprises bobbins 128 and collar 130. Bobbin 128 is preferably attached to collar 130 by fastener 132, which can optionally comprise a threaded fastener. Conductive wire 84 is disposed around bobbins 128.

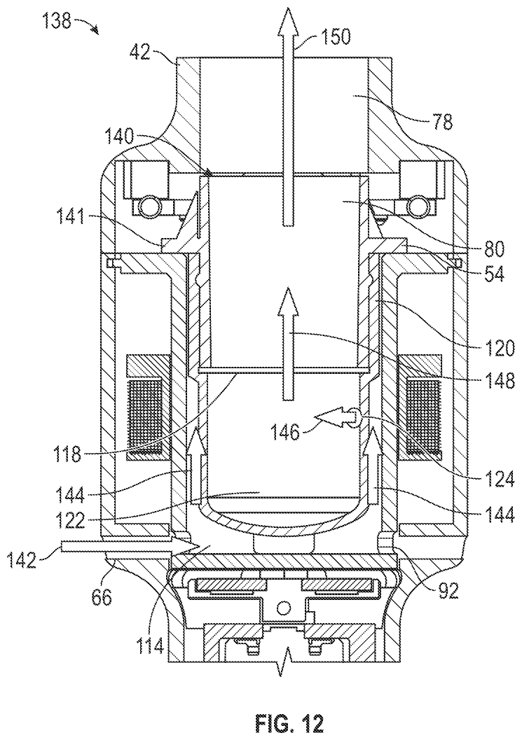

FIG. 12 illustrates air flow through a cutaway of a dry powder nebulizer 138. Air flow 142 enters dry powder nebulizer 138 through air inlet channel 66 and air inlet 92. Air flow 142 enters cavity 114 and air flow 146 enters cavity 122 of mixing compartment 120 by orifice 124. Regions 140 and 141 are sealed to prevent airflow out of dry powder nebulizer 138. Air flow 148 exits cavity 122 by traversing filter 118 and enters cavity 80 of mouthpiece adapter 54. Air flow 150 exits cavity 80 and enters cavity 78 of hinged top piece 42.

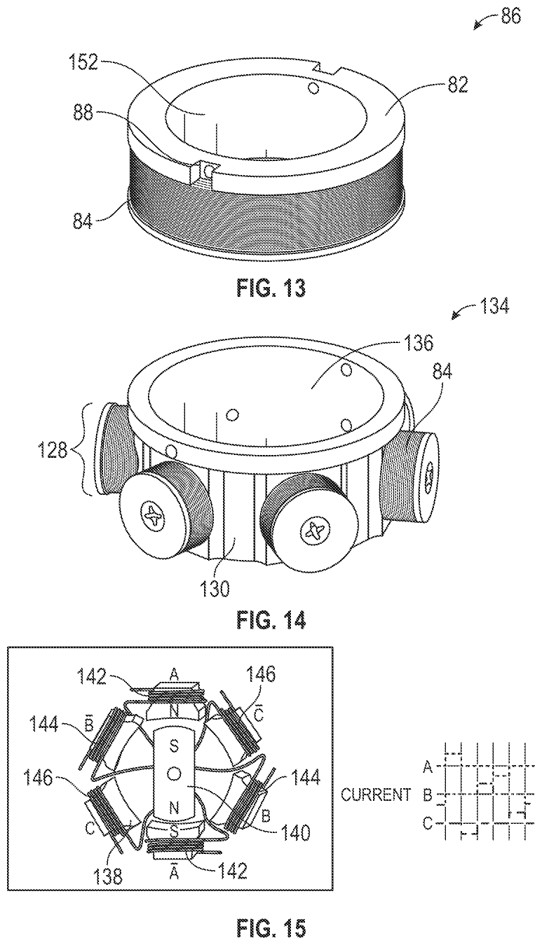

FIGS. 13 and 14 illustrate solenoid drive 86 and multi-coil multi-phase drive 134, respectively. Solenoid drive 86 comprises conductive wire 84 disposed around collar 82 and forms channel 152. Set screw 88, or another fastener or retention structure or substance can be disposed in collar 82. Multi-coil multi-phase drive 134 comprises bobbins 128 attached to collar 130. Conductive wire 84 is disposed around bobbins 128. Collar 130 forms channel 136. In one embodiment, solenoid drive 86 can be used in combination with multi-coil multi-phase drive 134—for example, solenoid drive 86 can be disposed above and/or below multi-coil multi-phase drive 134 and together the manipulation of current flow through solenoid drive 86 and multi-coil multi-phase drive 134 can be used to provide three dimensional movement of one or more MREs.

FIG. 15 shows a diagram of the agitation of an MRE within a multi-coil multi-phase drive. An MRE is disposed between pole pairs 142, 144, and 146. Pole pairs 142, 144, and 146 can be energized separately to move an MRE and are supported by scaffold 138.

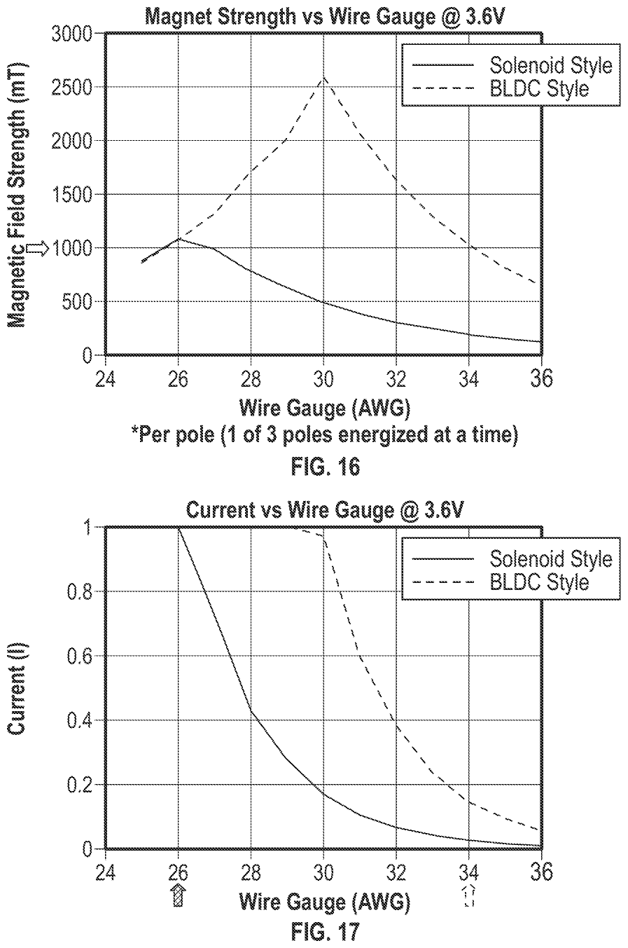

FIG. 16 shows magnetic strength at the wire coil surface vs. wire gauge for solenoid and multi-coil electro-magnet drive (labeled BLDC style) with a 3.6 volt excitation voltage. Magnetic field strength decreases with increasing wire gauge for the BLDC style drive, while magnetic field strength decreases as wire gauge increases or decreases below 30 AWG for the solenoid style drive.

FIG. 17 shows current vs. wire gauge for solenoid and multi-coil electro-magnet drive (labeled BLDC style) with a 3.6 volt excitation voltage. Current decreases with increasing wire gauge for both the BLDC and solenoid style drives.

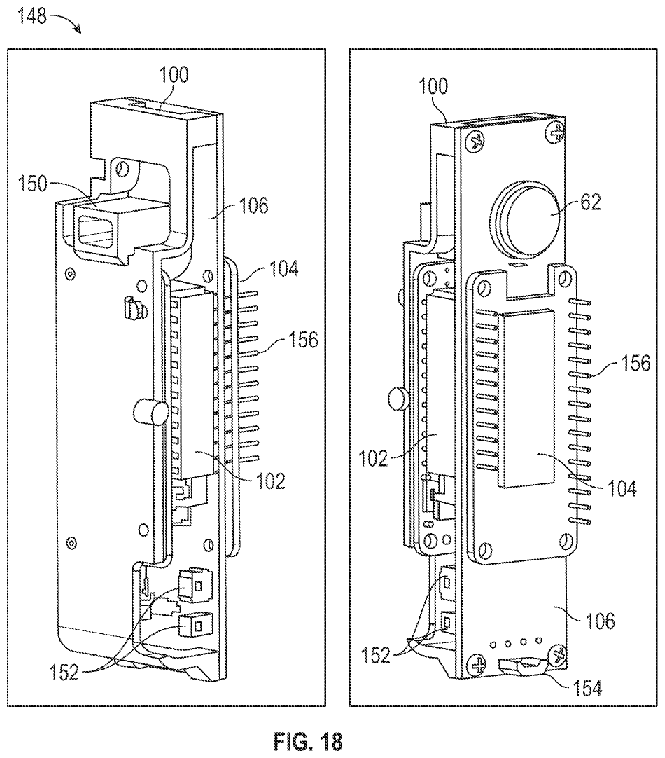

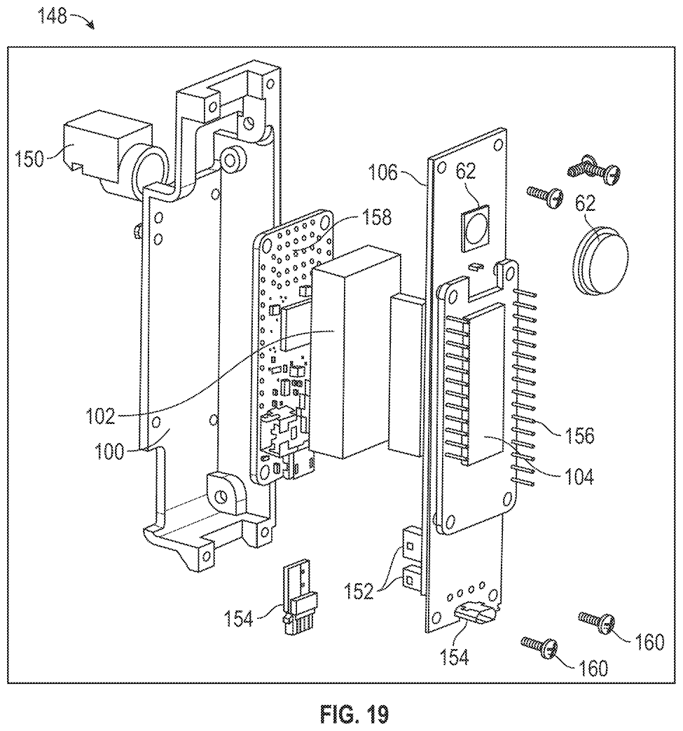

FIGS. 18 and 19 show the electronic components of a dry powder nebulizer 148 comprising a massless drive system. Circuit board 158, battery 102, and connectors 152 are disposed between inner chassis plate 100 and outer chassis plate 106. Conduit 150 exposes an electronic sensor to air flow through the cavity, for example a microphone or pressure sensor or other flow sensor. Headers 156 allow attachment of display 104 to outer chassis plate 106. USB connector 154 is attached to outer chassis plate 106. Inner chassis plate 100 and outer chassis plate 106 are connected by screws 160 or other fasteners or connecting mechanisms and/or structures.

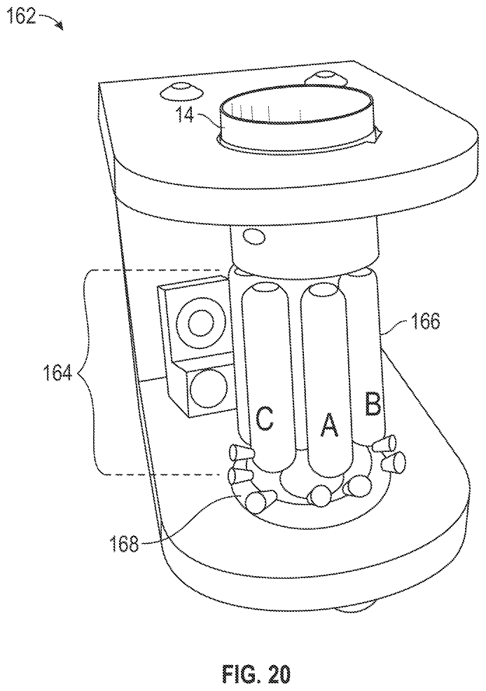

FIG. 20 shows massless mixing system 162 comprising mixing vessel 14 and multi-coil multi-phase massless drive 164. Massless drive 164 is disposed beneath mixing vessel 14 and comprises a plurality of conductive coils 166 and plate 168. Plate 168 provides charge to each of the conductive coils 166. Massless drive 164 is able to apply an axial or vertical electromagnetic field to an MRE at least partially disposed within mixing vessel 14.

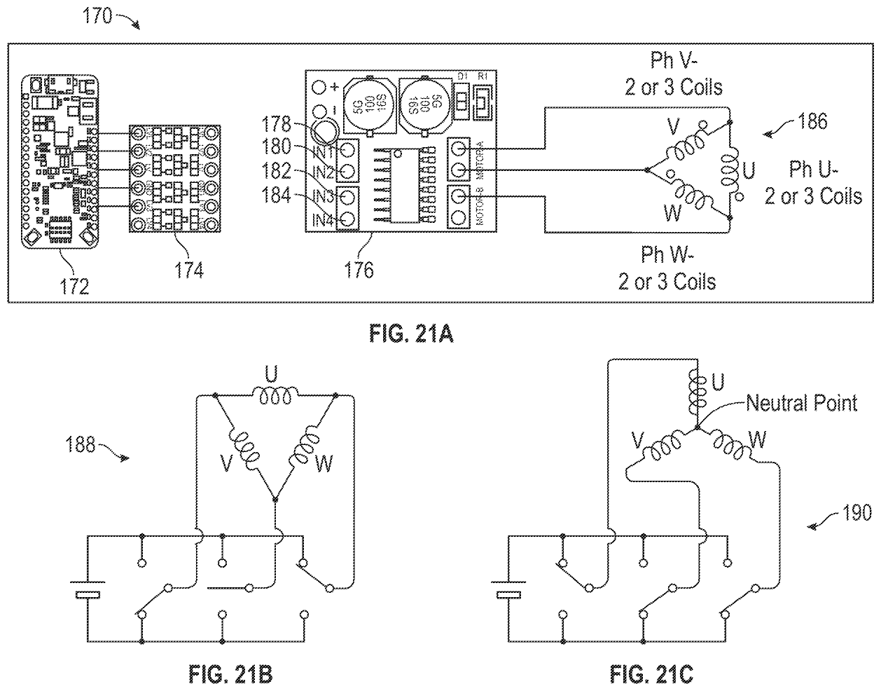

FIGS. 21A-21B show an embodiment of an excitation setup for a multi-coil electro-magnet drive massless mixing system wherein the driver setup comprises a 3-phase delta connection. FIG. 21C shows an alternative 3-phase star connection of the excitation setup for a multi-coil electro-magnet drive massless mixing system. Massless mixing system driver 170 comprising circuit 172, optional level shifter 174, and driver 176. Driver 176 comprises phase 1 input 178, phase 2 input 180, phase 3 input 182, optional input 184, and delta connection 186. Driver 176 may optionally comprise delta connection 188 or star connection 190.

The massless drive can be disposed below, around, or above the first housing. The massless drive can operate at a voltage of at least about 2 volts (“V”), about 2 V to about 12 V, about 3 V to about 11 V, about 4 V to about 10 V, about 5 V to about 9 V, about 6 V to about 8 V, or about 12 V, or at any other desired voltage. The massless drive can operate at about 3.3 V. The massless drive can operate with current of at least about least about 0.05 A, about 0.05 A to about 7.0 A, about 0.1 A to about 6.5 A, about 1.0 A to about 6.0 A, about 1.5 A to about 5.5 A, about 2.0 A to about 6.0 A, about 2.5 A to about 5.5 A, about 3.0 A to about 5.0 A, about 3.5 A to about 4.5 A, or about 7.0 A or more. The massless drive can operate at about 110 volts alternating current (“VAC”), about 110 VAC to about 220 VAC, about 120 VAC to about 210 VAC, about 130 VAC to about 200 VAC, about 140 VAC to about 190 VAC, about 150 VAC to about 180 VAC, about 160 VAC to about 170 VAC, or about 220 VAC or more. The massless drive can operate with direct current or with alternating current at a frequency of at least about 50 Hz, about 50 Hz to about 60 Hz, about 52 Hz to about 58 Hz, about 54 Hz to about 56 Hz, or about 60 Hz or more. The gauge of the conductive wire of the massless drive can be at least about 20 American wire gauge (“awg”), about 20 awg to about 30 awg, about 22 awg to about 28 awg, about 24 awg to about 26 awg, or about 30 awg or more. A larger diameter wire can be used to reduce resistance. The coil can be circular, or non-circular. The coil shape can shape the magnetic field generated. The core of the coils of the multi-coil multi-phase and/or solenoid can comprise an air core, an iron core, mild steel, stainless steel or another core material or combinations thereof.

The massless mixing system and/or dry powder nebulizer can comprise one or a plurality of massless drives. Each massless drive can receive periodic signals, i.e., electrical inputs, random or pseudo-random variations in signals. Optionally, each massless drive can be operated according to a preprogrammed routine. Each massless drive can receive a signal independent of another massless drive. The signal can vary in magnitude, sequence, and timing. The plurality of massless drives can receive a signal in phases.

The massless drive can be at least partially disposed below the first housing and/or the mixing vessel. The massless drive can direct an electromagnetic field in an upward direction relative to the massless drive. The massless drive can comprise a plate and one or more conductive coils. The conductive coil may comprise a conductive wire and/or other conductive material. The plate can be at least partially disposed beneath the one or more conductive coils and provide power to the one or more conductive coils.

The massless drive can comprise a solenoid drive, a multi-coil multi-phase drive, or a combination thereof or in combination with a permanent magnet drive. The solenoid drive can control the vertical movement of an MRE. The multi-coil multi-phase drive can control the horizontal, vertical, and chaotic movement of an MRE. The solenoid drive and multi-coil multi-phase drive can be disposed above and/or below one another. The solenoid drive and/or multi-coil multi-phase drive can comprise an electromagnet coil and a collar. The multi-coil multi-phase drive electromagnet can comprise a bobbin. The strength of the electromagnet coil can be estimated using the electromagnet formula (Equation 1) and the inverse square law (Equation 2) away from the face of the coil bobbins:

$$B = ( 1.257 * {10}^{⋀} - 6 * Ur * I * N ) / L , \qquad ( 1 )$$

$$B = 1 / {D}^{⋀} 2. \qquad ( 2 )$$

Relative permeability (Ur) is a function of the bobbin and surrounding material, which is an estimation. The electromagnet can comprise mild steel, 420 stainless steel, or a combination thereof. The mild steel can comprise a Ur of 10,000. The 420 stainless steel can comprise a Ur of at least about 40, about 40 to about 800, about 100 to about 700,about 200 to about 600, about 300 to about 500, or about 800 or more. The massless drive can comprise one or more electromagnet coils. From 1 to N coils can be concatenated and energized simultaneously. The one or more coils can be arranged in 2-N poles to create rotational positioning of magnetic fields to move an MRE. Each 2-N pole can be a phase, for example a six-coil massless drive is a three-phase massless drive. Each phase can be individually energized.

The first housing of the massless mixing system and/or dry powder nebulizer can comprise a cylindrical, hemi-spherical, tapered cylindrical, cuboid, or polygonal shape. The first housing can also comprise a soft formed container. The MRE of the massless mixing system and/or dry powder nebulizer can comprise PTFE or any other inert material. The MRE can also comprise a permanent magnet or magnetic material.

The massless mixing system and/or dry powder nebulizer can comprise electronics. The electronics can comprise an electronics chassis. The electronics chassis can comprise a chassis plate. The electronics can comprise a microphone and/or an air flow sensor. The microphone or other sensor can be attached to a chassis plate. The electronics can comprise one or more connectors. The connector can comprise a USB, electromagnet, motor, other connector, or a combination thereof. The USB can be a USB breakout. The electronics chassis can comprise one, two, three, or any number of boards to mount electronics.

The electronics can comprise an activation switch and/or activation button, an internal battery, a user interface, a circuit and/or circuit board, a driver, or a combination thereof including but not limited to memory, which can be user programmable and/or preprogrammed to activate one or more bobbins and/or solenoids of an embodiment of the present invention according to a predetermined or user-determined routine. The activation switch can be operated at a force of at least about 2 N, about 2 N to about 5 N, about 2.5 N to about 4.5 N, about 3 N to about 4 N, or about 5 N. The activation switch can be operated at a force of about 3.7 N. The internal battery can maintain a voltage of at least about 1 V, about 1 V to about 10 V, about 2 V to about 9 V, about 3 V to about 8 V, about 4 V to about 7 V, about 5 V to about 6 V, or about 9 V or more. The internal battery can maintain a voltage of about 3.7 V. The internal battery can comprise a charge of at least about 200 milliamp hour (“mAh”), about 200 mAh to about 600 mAh, about 250 mAh to about 550 mAh, about 300 mAh to about 500 mAh, about 350 mAh to about 450 mAh, about 300 mAh to about 400 mAh, or about 600 mAh. The user interface can comprise a display. The display can comprise a liquid crystal display, a light emitting diode (“LED”), including but not limited to an LED display, an organic LED, an e-ink display, or quantum dot LED display. The circuit and/or circuit board can comprise a microcontroller unit (“MCU”), a charge controller, other controller hardware, or a combination thereof. The driver can comprise a motor driver/feedback and/or an electromagnet driver. The electromagnet driver can comprise one or more channels. The electromagnet driver can generate a current of at least about 0.5 amps (“A”), about 0.5 A to about 7.0 A, about 1.0 A to about 6.5 A, about 1.5 A to about 6.0 A, about 2.0 A to about 5.5 A, about 2.5 A to about 5.0 A, about 3.0 A to about 4.5 A, about 3.5 A to about 4.0 A, or about 7.0 A or more.

The massless mixing system and/or dry powder nebulizer can comprise software. The software can be programmable. The software can control the flow of electricity through the massless mixing system and/or dry powder nebulizer. Specifically, the software can control the voltage, current, and/or power applied to the conductive wires of the massless mixing system and/or applied to the electronics of the massless mixing system and/or dry powder nebulizer. The software can control when and in what sequence voltage, current, and/or power is applied to individual coils of the multi-coil multi-phase or the single coil of solenoid drives of the massless mixing system. For example, the software can apply a current to a solenoid drive, then to two coils of the multi-coil multi-phase drives in a six-pole multi-coil multi-phase massless drive system. The software can comprise an artificial intelligence (“AI”), machine learning, compliance, or any other algorithm, or a combination thereof.

The massless mixing system and/or dry powder nebulizer can comprise a wireless communications module. The wireless communications module can comprise a Bluetooth Low Energy (“BLE”) unit. The wireless communications module can transmit data to and from the massless mixing system and/or dry powder nebulizer. The data can comprise profiles of massless drive for MRE movement within a housing. The data can comprise user data. The data can also comprise dose delivery control information, user adherence algorithms, artificial intelligence (“AI”) inference data, or a combination thereof. Data transmitted to and from the wireless communications module can be used to improve massless mixing systems and/or dry powder nebulizer performance and/or user outcomes.

The massless mixing system and/or dry powder nebulizer can generate a magnetic field. The magnetic field can comprise a rotating magnetic field. The rotating direction can be of consistent direction or of alternating direction. The magnetic field can comprise an intermittent magnetic field, for example to induce a disruption in the MRE motion. The magnetic field can be controlled to induce controlled and/or chaotic motion of one or more MRE. The magnetic field can control the motion of the MRE by magnetic coupling of attraction or repulsion. The magnetic field can be regulated or pseudo-random magnetic and have one or more field strength profiles. A field strength profile comprises the direction, strength, and modulation of a magnetic field. The magnitude of the magnetic field can be at least about 3 Gauss, about 3 Gauss to about 30,000 Gauss; about 30 Gauss to about 20,000 Gauss, about 300 Gauss to about 10,000 Gauss, about 3,000 Gauss to about 5,000 Gauss, or about 30,000 Gauss or more. Each field strength profile results in an MRE motion profile. The magnetic field can be rotated at a rate of at least about 100 rpm, about 100 rpm to about 5,000 rpm, about 500 rpm to about 4,500 rpm, about 1,000 rpm to about 4,000 rpm, about 1,500 rpm to about 3,500 rpm, about 2,000 rpm to about 3,000 rpm, or about 5,000 rpm or more. The magnetic field can be modulated to cause the magnetic field to rotate.

The massless mixing system and/or dry powder nebulizer can mix material by moving the MRE. The motion of the MRE can agitate dry powder to enhance dry powder emission from the mixing system and/or dry powder nebulizer. Enhanced dry powder emission during user inhalation treatment can improve medication delivery to the lung from the dry powder nebulizer. In some embodiments, the massless mixing system and/or dry powder nebulizer can move the MRE to prevent contact with the first housing. In some embodiments, the dry powder nebulizer can move the MRE to contact the filter with the MRE to prevent filter clogging, enhance powder dispersing, or other beneficial functions.

The massless drive can rotate the MRE without a rotating mass, i.e., a magnet, motor shaft, and rotor, other than the MRE. The massless drive can rotate the MRE around an axis and in a clockwise (“CW”) or counter-clockwise (“CCW”) direction. The massless drive can move the MRE in any direction and rapidly switch the direction of the MRE. The massless drive can change the direction of MRE in at least about 0.01 seconds (“sec”), about 0.01 sec to about 10 sec, about 0.1 sec to about 9 sec, about 0.5 sec to about 8 sec, about 1 sec to about 7 sec, about 2 sec to about 8 sec, about 3 sec to about 7 sec, about 4 sec to about 6, or about 10 sec or more. The massless drive can change the direction of MRE in about 0.7 sec. The massless drive can move to MRE to create a motion profile. The massless drive of the dry powder nebulizer can create an MRE motion profile specific to the powdered medication and/or user characteristics. For example, specific motion profile can be needed if the medication requires extensive nebulization to generate a fine aerosol or if the user has a respiratory condition. The massless drive of the dry powder nebulizer can also create an MRE motion profile to cause MRE impacts with the filter independently from MRE mixing of material in the first housing.

Elimination of a rotating mass by the massless drive improves device operation relative to mass drives comprising a permanent bar magnet. These improvements include, but are not limited to, generating a rotating magnetic field using only electronic hardware without or with software control, reducing energy consumption, preventing material, e.g., powder, penetration into massless drive system and/or dry powder nebulizer; and improved device packaging due to sealing for cleaning and less volume required for the massless drive.

The massless drive also resolves other drawbacks of a mass drive, including, but not limited to, the limitation on magnetic field variation from a fixed-mount permanent magnet and a DC motor; the limitation on magnetic field variation due to mass inertia of permanent magnet rotated by the DC motor; and variation in performance of the mechanical system due to fabrication and manufacturing tolerances and wear of mechanical components.

Optionally, the massless mixing system and/or dry powder nebulizer can comprise a permanent bar magnet and/or a motor. The permanent bar magnet can be magnetically coupled to the MRE. The permanent bar magnet can be mounted on a shaft of the motor with the bar magnet at least partially disposed beneath the first housing. The motor can be a DC or AC motor.

The dry powder nebulizer can have an airflow through the device during its operation. The airflow can enter the dry powder nebulizer through the air inlet. The airflow can surround the first housing and enter the first housing through the second air inlet. The airflow can traverse across the filter and exit out of the dry powder nebulizer.

Optionally, embodiments of the present invention can include a general or specific purpose computer or distributed system programmed with computer software implementing steps described above, which computer software can be in any appropriate computer language, including but not limited to C++, FORTRAN, BASIC, Java, Python, Linux, assembly language, microcode, distributed programming languages, etc. The apparatus can also include a plurality of such computers/distributed systems (e.g., connected over the Internet and/or one or more intranets) in a variety of hardware implementations. For example, data processing can be performed by an appropriately programmed microprocessor, computing cloud, Application Specific Integrated Circuit (ASIC), Field Programmable Gate Array (FPGA), or the like, in conjunction with appropriate memory, network, and bus elements. One or more processors and/or microcontrollers can operate via instructions of the computer code and the software is preferably stored on one or more tangible non-transitive memory-storage devices.

Note that in the specification and claims, “about” or “approximately” means within twenty percent (20%) of the amount or value given. All computer software disclosed herein can be embodied on any non-transitory computer-readable medium (including combinations of mediums), including without limitation CD-ROMs, DVD-ROMs, hard drives (local or network storage device), USB keys, other removable drives, ROM, and firmware.

Although the invention has been described in detail with particular reference to the disclosed embodiments, other embodiments can achieve the same results. Variations and modifications of the present invention will be obvious to those skilled in the art and it is intended to cover all such modifications and equivalents. The entire disclosures of all references, applications, patents, and publications cited above and/or in the attachments, and of the corresponding application(s), are hereby incorporated by reference. Unless specifically stated as being “essential” above, none of the various components or the interrelationship thereof are essential to the operation of the invention. Rather, desirable results can be achieved by substituting various components and and/or reconfiguration of their relationships with one another.

What is claimed is:

1. A massless mixing system comprising:
a power source;
a mixing vessel;
a magnetic responsive element at least partially disposed within said mixing vessel; and
a massless drive in communication with said mixing vessel, wherein said massless drive comprises:
a collar forming a channel; and
a conductive wire at least partially disposed around said collar.

2. The massless mixing system of claim 1 wherein said massless drive applies an electromagnetic field onto said mixing vessel.

3. The massless mixing system of claim 1 wherein said massless drive is at least partially disposed around said mixing vessel.

4. The massless mixing system of claim 1 wherein said massless drive is at least partially disposed beneath said mixing vessel.

5. The massless mixing system of claim 1 wherein said massless drive comprises a solenoid.

6. The massless mixing system of claim 1 wherein said massless drive comprises a multi-coil multi-phase drive.

7. The massless mixing system of claim 1 wherein said massless drive comprises a plurality of massless drives controlling a vertical and a horizontal movement of said magnetic responsive element.

8. The massless mixing system of claim 1 further comprising a circuit.

9. The massless mixing system of claim 8 wherein said circuit controls a power supplied to said massless drive.

10. The massless mixing system of claim 1 further comprising a first housing at least partially disposed around said massless drive.

11. The massless mixing system of claim 1 further comprising a second housing at least partially disposed above said mixing vessel.

12. The massless mixing system of claim 1 further comprising a filter.

13. The massless mixing system of claim 12 wherein said filter is disposed above said mixing vessel.

14. The massless mixing system of claim 1 further comprising a programmable software to control a flow of electricity through said massless mixing system.

15. A method of massless mixing, the method comprising:
providing a material to be mixed;
at least partially disposing a portion of the material into a mixing vessel;
contacting the material with a magnetic responsive element;
applying power to a massless drive;
applying power to the massless drive; and
applying an electromagnetic field to the magnetic responsive element.

16. The method of claim 15 further comprising contacting the material with a filter.

17. The method of claim 15 further comprising rotating the magnetic responsive element.

18. The method of claim 15 further comprising moving the magnetic responsive element in a vertical direction.

19. The method of claim 15 further comprising moving the magnetic responsive element in a horizontal direction.

20. The method of claim 15 wherein the electromagnetic field comprises a plurality of electromagnetic fields each applying a different force.
